(19) United States
(12) Patent Application Publication
(10) Pub. No.: US 2023/0338623 A1
(43) Pub. Date: Oct. 26, 2023

(54) MEDICAL DEVICE COATINGS WITH MICROCRYSTALLINE ACTIVE AGENTS

(71) Applicant: Surmodics, Inc., Eden Prairie, MN (US)

(72) Inventors: Joram Slager, St. Louis Park, MN (US); Michael Schneider, Minneapolis, MN (US); Sedef Piril Ertem, Minneapolis, MN (US); Travis Lemke, Chanhassen, MN (US); Ralph A. Chappa, Ham Lake, MN (US)

(21) Appl. No.: 18/139,125

(22) Filed: Apr. 25, 2023

Related U.S. Application Data
(60) Provisional application No. 63/334,497, filed on Apr. 25, 2022.
(60) Provisional application No. 63/414,710, filed on Oct. 10, 2022.

(51) Int. Cl. A61L 29/16; A61L 29/08

(52) U.S. Cl. CPC A61L 29/16; A61L 29/085; A61L 2300/216; A61L 2300/802; A61L 2300/416

(57) ABSTRACT

Embodiments herein relate to medical devices and coatings for the same. In an embodiment, a drug delivery coating can be included having a polymeric layer. The polymeric layer can include a hydrophilic outer surface. The drug delivery coating can also include an active agent layer disposed over the polymeric layer. The active agent layer can include a microcrystalline active agent and a cationic agent. Other embodiments are also included herein.

This application claims the benefit of U.S. Provisional Application No. 63/334,497, filed Apr. 25, 2022, and U.S. Provisional Application No. 63/414,710, filed Oct. 10, 2022, the content of both of which is herein incorporated by reference in their entirety.

### FIELD

Embodiments herein relate to medical devices and coatings for the same.

### BACKGROUND

The human vascular system is subject to blockage due to plaque within the arteries. Partial and even complete blockage of arteries by the formation of an atherosclerotic plaque is a well-known and frequent medical problem. Frequently, such blockage occurs in the coronary arteries. However, blockages may also occur secondary to past treatment of specific sites (restenosis—such as that stemming from rapidly dividing smooth muscle cells). In addition, blockages can also occur in the context of peripheral arteries.

One common procedure for the treatment of blocked arteries is percutaneous transluminal coronary angioplasty (PTCA), also referred to as balloon angioplasty. In this procedure, a catheter having an inflatable balloon at its distal end is introduced into the coronary artery, the deflated, folded balloon is positioned at the stenotic site, and then the balloon is inflated. Inflation of the balloon disrupts and flattens the plaque against the arterial wall, and stretches the arterial wall, resulting in enlargement of the intraluminal passageway and increased blood flow. After such expansion, the balloon is deflated, and the balloon catheter removed. A similar procedure, called percutaneous transluminal angioplasty (PTA), is used in arteries other than coronary arteries in the vascular system. In other related procedures, a small mesh tube, referred to as a stent is implanted at the stenotic site to help maintain patency of the coronary artery, preventing mechanical recoil. In rotoblation procedures, also called percutaneous transluminal rotational atherectomy (PCRA), a small, diamond-tipped, drill-like device is inserted into the affected artery by a catheterization procedure to remove fatty deposits or plaque. In a cutting balloon procedure, a balloon catheter with small blades is inflated to position the blades, score the plaque and compress the fatty matter into the artery wall.

During one or more of these procedures, it may be desirable to also deliver a therapeutic agent or drug to the area where the treatment is occurring to prevent restenosis, repair vessel dissections or small aneurysms or provide other desired therapy. Additionally, it may be desirable to transfer therapeutic agents to other locations in a mammal, such as the skin, neurovasculature, nasal, oral, the lungs, the mucosa, sinus, the GI tract or the renal peripheral vasculature.

### SUMMARY

Embodiments herein relate to medical devices and coatings for the same. In a first aspect, a drug delivery coating can be included having a polymeric layer. The polymeric layer can include a hydrophilic outer surface. The drug delivery coating can also include an active agent layer disposed over the polymeric layer. The active agent layer can include a microcrystalline active agent and a cationic agent.

In a second aspect, in addition to one or more of the preceding or following aspects, or in the alternative to some aspects, the microcrystalline active agent can have an average particle size of less than 50 μm.

In a third aspect, in addition to one or more of the preceding or following aspects, or in the alternative to some aspects, the microcrystalline active agent can have an average particle size of less than 20 μm.

In a fourth aspect, in addition to one or more of the preceding or following aspects, or in the alternative to some aspects, the microcrystalline active agent can be at least 90 percent crystalline.

In a fifth aspect, in addition to one or more of the preceding or following aspects, or in the alternative to some aspects, the microcrystalline active agent can be at least 95 percent crystalline.

In a sixth aspect, in addition to one or more of the preceding or following aspects, or in the alternative to some aspects, the total amount of amorphous active agent in the active agent layer can be less than 10% by weight.

In a seventh aspect, in addition to one or more of the preceding or following aspects, or in the alternative to some aspects, the total amount of amorphous active agent in the active agent layer can be less than 5% by weight.

In an eighth aspect, in addition to one or more of the preceding or following aspects, or in the alternative to some aspects, the microcrystalline active agent can include sirolimus.

In a ninth aspect, in addition to one or more of the preceding or following aspects, or in the alternative to some aspects, the cationic agent can include at least one selected from the group consisting of polyethyleneimine (PEI), 1,2-dioleoyl-3-trimethylammonium-propane (DOTAP), and polyamidoamine dendrimers (PAMAM).

In a tenth aspect, in addition to one or more of the preceding or following aspects, or in the alternative to some aspects, the microcrystalline active agent can be arranged leaving gaps between some adjacent crystals and the cationic agent fills some of the gaps.

In an eleventh aspect, in addition to one or more of the preceding or following aspects, or in the alternative to some aspects, some gaps remain unfilled.

In a twelfth aspect, in addition to one or more of the preceding or following aspects, or in the alternative to some aspects, the cationic agent coats at least some crystals of the microcrystalline active agent.

In a thirteenth aspect, in addition to one or more of the preceding or following aspects, or in the alternative to some aspects, the polymeric layer further can include a hydrophilic polymer.

In a fourteenth aspect, in addition to one or more of the preceding or following aspects, or in the alternative to some aspects, the hydrophilic polymer can include at least one selected from the group consisting of a methacrylamide and a polyvinylpyrrolidone.

In a fifteenth aspect, in addition to one or more of the preceding or following aspects, or in the alternative to some aspects, the hydrophilic polymer can include a methacrylamide copolymer.

In a sixteenth aspect, in addition to one or more of the preceding or following aspects, or in the alternative to some aspects, the hydrophilic polymer can include a photoreactive methacrylamide copolymer.

In a seventeenth aspect, in addition to one or more of the preceding or following aspects, or in the alternative to some aspects, the hydrophilic polymer can include poly[N-(3-aminopropyl)methacrylamide-co-N-(3-(4-benzoylbenazmido)propyl)methacrylamide].

In an eighteenth aspect, in addition to one or more of the preceding or following aspects, or in the alternative to some aspects, the polymeric layer further can include a crosslinking agent.

In a nineteenth aspect, in addition to one or more of the preceding or following aspects, or in the alternative to some aspects, the polymeric layer further can include a photoreactive crosslinking agent.

In a twentieth aspect, in addition to one or more of the preceding or following aspects, or in the alternative to some aspects, the photoreactive crosslinking agent can include benzophenone groups.

In a twenty-first aspect, in addition to one or more of the preceding or following aspects, or in the alternative to some aspects, the photoreactive crosslinking agent can include ethylenebis (4-benzoylbenzyldimethylammonium) dibromide.

In a twenty-second aspect, in addition to one or more of the preceding or following aspects, or in the alternative to some aspects, the polymeric layer further can include a methacrylamide.

In a twenty-third aspect, in addition to one or more of the preceding or following aspects, or in the alternative to some aspects, the polymeric layer further can include a polyvinylpyrrolidone.

In a twenty-fourth aspect, in addition to one or more of the preceding or following aspects, or in the alternative to some aspects, the active agent layer further can include an additive.

In a twenty-fifth aspect, in addition to one or more of the preceding or following aspects, or in the alternative to some aspects, the additive can include at least one selected from the group consisting of glycogen, dextran, and F68 poloxamer.

In a twenty-sixth aspect, a drug delivery device can be included having a substrate and a polymeric layer. The polymeric layer can include a hydrophilic outer surface. The polymeric layer can be disposed over the substrate. The drug delivery device can also include an active agent layer. The active agent layer can be disposed over the polymeric layer. The active agent layer can include a microcrystalline active agent and a cationic agent.

In a twenty-seventh aspect, in addition to one or more of the preceding or following aspects, or in the alternative to some aspects, the microcrystalline active agent can have an average particle size of less than 50 μm.

In a twenty-eighth aspect, in addition to one or more of the preceding or following aspects, or in the alternative to some aspects, the microcrystalline active agent can have an average particle size of less than 20 μm.

In a twenty-ninth aspect, in addition to one or more of the preceding or following aspects, or in the alternative to some aspects, the microcrystalline active agent can be at least 90 percent crystalline.

In a thirtieth aspect, in addition to one or more of the preceding or following aspects, or in the alternative to some aspects, the microcrystalline active agent can be at least 95 percent crystalline.

In a thirty-first aspect, in addition to one or more of the preceding or following aspects, or in the alternative to some aspects, the total amount of amorphous active agent in the active agent layer can be less than 10% by weight.

In a thirty-second aspect, in addition to one or more of the preceding or following aspects, or in the alternative to some aspects, the total amount of amorphous active agent in the active agent layer can be less than 5% by weight.

In a thirty-third aspect, in addition to one or more of the preceding or following aspects, or in the alternative to some aspects, the microcrystalline active agent can include sirolimus.

In a thirty-fourth aspect, in addition to one or more of the preceding or following aspects, or in the alternative to some aspects, the cationic agent can include at least one selected from the group consisting of polyethyleneimine (PEI), 1,2-dioleoyl-3-trimethylammonium-propane (DOTAP), and polyamidoamine dendrimers (PAMAM).

In a thirty-fifth aspect, in addition to one or more of the preceding or following aspects, or in the alternative to some aspects, the microcrystalline active agent can be arranged leaving gaps between some adjacent crystals and the cationic agent fills some of the gaps.

In a thirty-sixth aspect, in addition to one or more of the preceding or following aspects, or in the alternative to some aspects, some gaps remain unfilled.

In a thirty-seventh aspect, in addition to one or more of the preceding or following aspects, or in the alternative to some aspects, the cationic agent coats at least some crystals of the microcrystalline active agent.

In a thirty-eighth aspect, in addition to one or more of the preceding or following aspects, or in the alternative to some aspects, the polymeric layer further can include a hydrophilic polymer.

In a thirty-ninth aspect, in addition to one or more of the preceding or following aspects, or in the alternative to some aspects, the hydrophilic polymer can include at least one selected from the group consisting of a methacrylamide and a polyvinylpyrrolidone.

In a fortieth aspect, in addition to one or more of the preceding or following aspects, or in the alternative to some aspects, the hydrophilic polymer can include a methacrylamide copolymer.

In a forty-first aspect, in addition to one or more of the preceding or following aspects, or in the alternative to some aspects, the hydrophilic polymer can include a photoreactive methacrylamide copolymer.

In a forty-second aspect, in addition to one or more of the preceding or following aspects, or in the alternative to some aspects, the hydrophilic polymer can include poly[N-(3-aminopropyl)methacrylamide-co-N-(3-(4-benzoylbenazmido)propyl)methacrylamide].

In a forty-third aspect, in addition to one or more of the preceding or following aspects, or in the alternative to some aspects, the polymeric layer further can include a crosslinking agent.

In a forty-fourth aspect, in addition to one or more of the preceding or following aspects, or in the alternative to some aspects, the polymeric layer further can include a photoreactive crosslinking agent.

In a forty-fifth aspect, in addition to one or more of the preceding or following aspects, or in the alternative to some aspects, the photoreactive crosslinking agent can include benzophenone groups.

In a forty-sixth aspect, in addition to one or more of the preceding or following aspects, or in the alternative to some aspects, the photoreactive crosslinking agent can include ethylenebis (4-benzoylbenzyldimethylammonium) dibromide.

In a forty-seventh aspect, in addition to one or more of the preceding or following aspects, or in the alternative to some aspects, the polymeric layer further can include a methacrylamide.

In a forty-eighth aspect, in addition to one or more of the preceding or following aspects, or in the alternative to some aspects, the polymeric layer further can include a polyvinylpyrrolidone.

In a forty-ninth aspect, in addition to one or more of the preceding or following aspects, or in the alternative to some aspects, the active agent layer further can include an additive.

In a fiftieth aspect, in addition to one or more of the preceding or following aspects, or in the alternative to some aspects, the additive can include at least one selected from the group consisting of glycogen, dextran, and F68 poloxamer.

In a fifty-first aspect, in addition to one or more of the preceding or following aspects, or in the alternative to some aspects, the substrate can include a polymer and the substrate forms part of an expandable balloon.

In a fifty-second aspect, in addition to one or more of the preceding or following aspects, or in the alternative to some aspects, the drug delivery device can be a drug delivery balloon catheter.

In a fifty-third aspect, a drug delivery device can be included having a substrate and an active agent layer. The active agent layer can be disposed over the substrate. The active agent layer can include a microcrystalline active agent and a cationic agent.

In a fifty-fourth aspect, in addition to one or more of the preceding or following aspects, or in the alternative to some aspects, the microcrystalline active agent can have an average particle size of less than 50 μm.

In a fifty-fifth aspect, in addition to one or more of the preceding or following aspects, or in the alternative to some aspects, the microcrystalline active agent can have an average particle size of less than 20 μm.

In a fifty-sixth aspect, in addition to one or more of the preceding or following aspects, or in the alternative to some aspects, the microcrystalline active agent can be at least 90 percent crystalline.

In a fifty-seventh aspect, in addition to one or more of the preceding or following aspects, or in the alternative to some aspects, the microcrystalline active agent can be at least 95 percent crystalline.

In a fifty-eighth aspect, in addition to one or more of the preceding or following aspects, or in the alternative to some aspects, the total amount of amorphous active agent in the active agent layer can be less than 10% by weight.

In a fifty-ninth aspect, in addition to one or more of the preceding or following aspects, or in the alternative to some aspects, the total amount of amorphous active agent in the active agent layer can be less than 5% by weight.

In a sixtieth aspect, in addition to one or more of the preceding or following aspects, or in the alternative to some aspects, the microcrystalline active agent can include sirolimus.

In a sixty-first aspect, in addition to one or more of the preceding or following aspects, or in the alternative to some aspects, the cationic agent can include at least one selected from the group consisting of polyethyleneimine (PEI), 1,2-dioleoyl-3-trimethylammonium-propane (DOTAP), and polyamidoamine dendrimers (PAMAM).

In a sixty-second aspect, in addition to one or more of the preceding or following aspects, or in the alternative to some aspects, wherein the microcrystalline active agent can be arranged leaving gaps between some adjacent crystals, and wherein the cationic agent fills some of the gaps.

In a sixty-third aspect, in addition to one or more of the preceding or following aspects, or in the alternative to some aspects, wherein some gaps remain unfilled.

In a sixty-fourth aspect, in addition to one or more of the preceding or following aspects, or in the alternative to some aspects, the cationic agent coats at least some crystals of the microcrystalline active agent.

In a sixty-fifth aspect, in addition to one or more of the preceding or following aspects, or in the alternative to some aspects, the active agent layer further can include a crosslinking agent.

In a sixty-sixth aspect, in addition to one or more of the preceding or following aspects, or in the alternative to some aspects, the active agent layer further can include a photoreactive crosslinking agent.

In a sixty-seventh aspect, in addition to one or more of the preceding or following aspects, or in the alternative to some aspects, the photoreactive crosslinking agent can include benzophenone groups.

In a sixty-eighth aspect, in addition to one or more of the preceding or following aspects, or in the alternative to some aspects, the photoreactive crosslinking agent can include ethylenebis (4-benzoylbenzyldimethylammonium) dibromide.

In a sixty-ninth aspect, in addition to one or more of the preceding or following aspects, or in the alternative to some aspects, the active agent layer further can include an additive.

In a seventieth aspect, in addition to one or more of the preceding or following aspects, or in the alternative to some aspects, the additive can include at least one selected from the group consisting of glycogen, dextran, and F68 poloxamer.

In a seventy-first aspect, in addition to one or more of the preceding or following aspects, or in the alternative to some aspects, the substrate can include a polymer, wherein the substrate forms part of an expandable balloon.

In a seventy-second aspect, in addition to one or more of the preceding or following aspects, or in the alternative to some aspects, the drug delivery device can be a drug delivery balloon catheter.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a coated medical device in accordance with various embodiments herein.

FIG. 2 is a schematic cross-sectional view of a coating as taken along line 2-2′ of FIG. 1 in accordance with various embodiments herein.

FIG. 3 is a schematic cross-sectional view of an alternative coating as taken along line 2-2′ of FIG. 1 in accordance with various embodiments herein.

FIG. 4 is a schematic cross-sectional view of a coating in accordance with various embodiments herein.

FIG. 5 is a schematic cross-sectional view of a coating in accordance with various embodiments herein.

FIG. 6 is a schematic cross-sectional view of a portion of an active agent layer of a coating in accordance with various embodiments herein.

FIG. 7 is a schematic cross-sectional view of a portion of an active agent layer of a coating in accordance with various embodiments herein.

FIG. 8 is a diagram illustrating the drug delivery of the active agent over the length of the balloon delivered from a drug delivery coating prepared in accordance with various embodiments herein.

FIG. 9 is a diagram illustrating the active agent retained on the balloon after tracking through a simulated blood vessel from a drug delivery coating prepared in accordance with various embodiments compared against as two commercial drug delivery coatings.

FIG. 10 is a diagram illustrating the effective active agent in retained on or in the tissue wall as a factor of time delivered from a drug delivery coating prepared in accordance with various embodiments herein compared against as two commercial drug delivery coatings.

FIG. 11 is a diagram illustrating the occurrence of restenosis in patients treated with a drug delivery coating prepared in accordance with various embodiments herein compared against two commercial drug delivery coatings and two control coating.

FIG. 12 is a diagram illustrating the histopathology biomarkers indicating active agent effect in the tissue after 30 days with active agent delivered from a drug delivery coating prepared in accordance with various embodiments herein compared against one commercial drug delivery coating.

FIG. 13 is a diagram illustrating the histopathology biomarkers indicating active agent effect in the tissue after 30 days with active agent delivered from a drug delivery coating prepared in accordance with various embodiments herein compared against one commercial drug delivery coating.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

It can be desirable to deliver a therapeutic agent, drug, or active agent to an area of the vasculature to prevent restenosis, repair vessel dissections or small aneurysms, or to provide other desired therapy. In general, it is advantageous to transfer a therapeutic amount of the therapeutic agent or drug transferred to the targeted treatment area and maintaining sufficient therapeutic agent and drug in sufficient amounts to have a therapeutic effect after the initial treatment. In particular, restenosis can occur days or months after the initial treatment. Accordingly, anti-restenosis agents or drugs must be transferred in a sufficiently high initial dose to have an immediate therapeutic effect as well as remain on the vessel wall with for later absorption into the tissue to have a prolonged therapeutic effect. Embodiments herein include medical devices and coatings for the same including microcrystalline active agents exhibiting high levels of transfer and retention by targeted tissue.

In an embodiment, a drug delivery coating is included herein including a polymeric layer, the polymeric layer comprising a hydrophilic outer surface. The drug delivery coating also includes an active agent layer, wherein the active agent layer is disposed over the polymeric layer. The active agent layer can include a microcrystalline active agent and a cationic agent for improving selective retention of the active agent on the delivery device and maintaining the active agent on the vessel wall for absorption into the tissue.

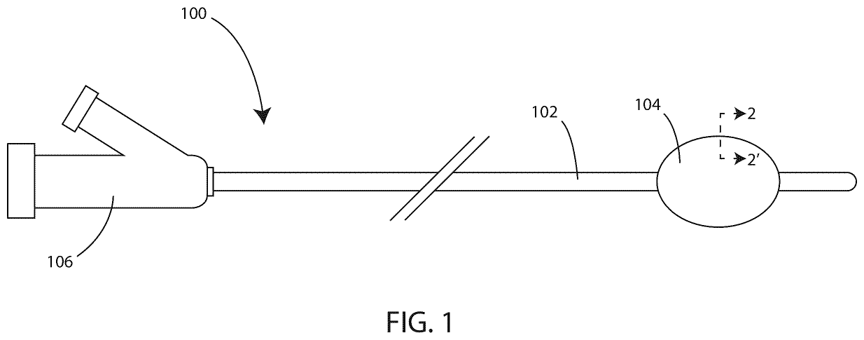

Referring now to FIG. 1, a schematic view of a coated medical device 100 is shown in accordance with various embodiments herein. In various embodiments, the medical device 100 can, specifically, be a balloon catheter. In this example, the medical device 100 includes a proximal manifold 106. The medical device 100 also includes a shaft 102. The medical device 100 also includes a balloon 104. The balloon 104 of the medical device 100 can be coated with a drug delivery coating whereby active agent is released from the balloon 104 upon expansion of the balloon 104. In some embodiments, the shaft 102 of the medical device 100 can be coated with a lubricious coating.

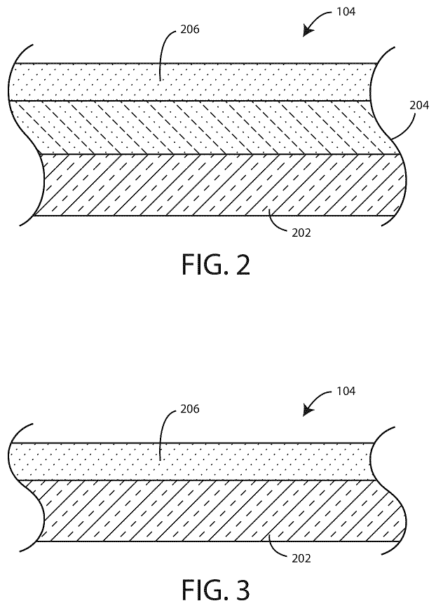

Referring now to FIG. 2, a schematic cross-sectional view is shown of a coating as taken along line 2-2′ of FIG. 1 in accordance with various embodiments herein. The balloon 104 includes a substrate 202 and a polymer layer 204 (such as a hydrophilic polymer layer) and an active agent layer 206. In some embodiments, the active agent layer 206 can be disposed over the polymer layer 204. However, in some embodiments, such as shown herein the active agent layer 206 can be layered directly on the substrate 202. The active agent layer 206 can include various components such as a microcrystalline active agent and a cationic agent as described in greater detail below.

Referring now to FIG. 3, a schematic cross-sectional view is shown of a coating as taken along line 2-2′ of FIG. 1 in accordance with various embodiments herein. FIG. 3 is generally similar to FIG. 2. However, in this embodiment, the intermediate polymer layer 204 is omitted. In some embodiments, the substrate 202 can be treated to alter the surface texture and the surface energy thereof such as by corona treatment, plasma treatment, chemical treatment or the like. The altered surface energy can interact with the cationic agent, microcrystalline active agent, intermediate polymer layer 204, and combinations thereof.

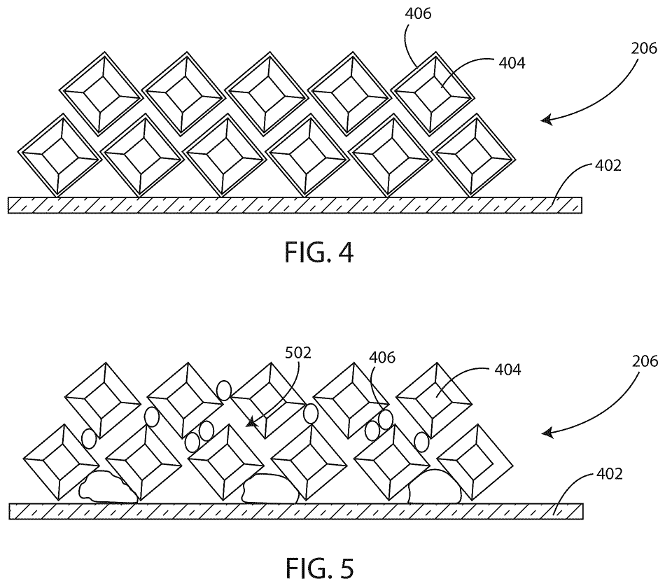

Referring now to FIG. 4, a schematic cross-sectional view is shown of a coating in accordance with various embodiments herein. In this view, the active agent layer 206 is shown as disposed on an intermediate layer 402 (which could be a substrate, a hydrophilic polymer layer, or another type of layer). The active agent layer 206 is shown to include a plurality of active agent crystals 404 and a cationic agent 406 applied to the active agent crystals 404 for retaining the active agent crystals 404 to the substrate 202, intermediate layer 402, adjacent crystals 404, or combinations thereof. In this example, a cationic agent 406 is shown coating surfaces of the crystals 404. The cationic agent 406 can partially coat the crystals 404 to form patches of cationic agent 406 on the surface of the crystals 404 or entirely coat the crystals 404 to encapsulate the crystals 404 with the cationic agent 406.

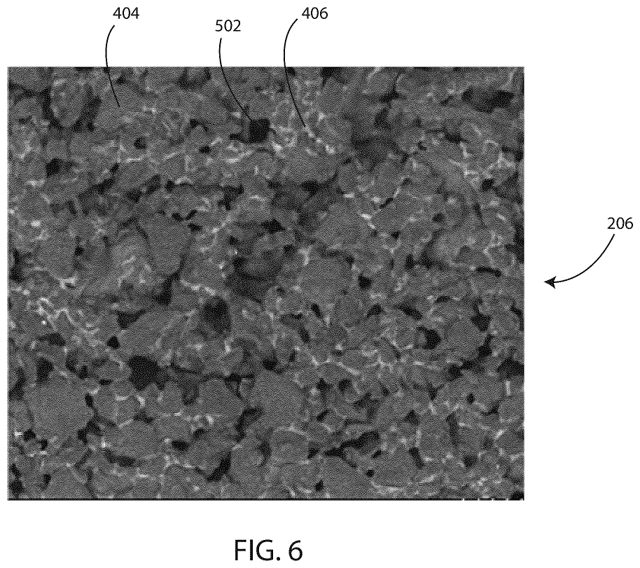

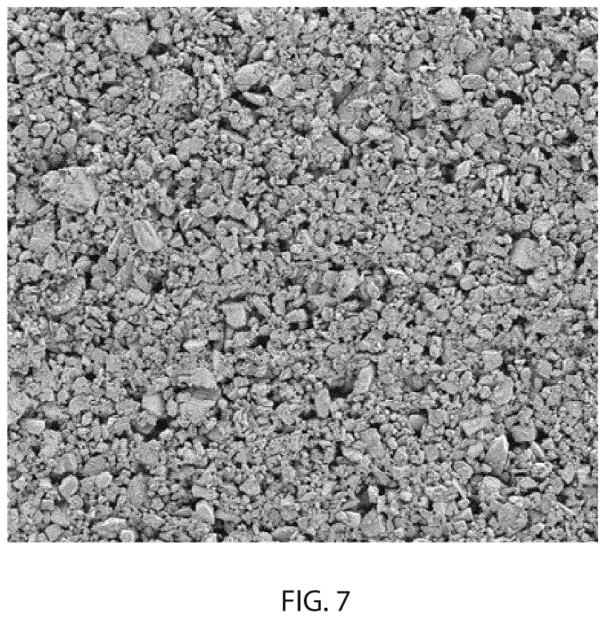

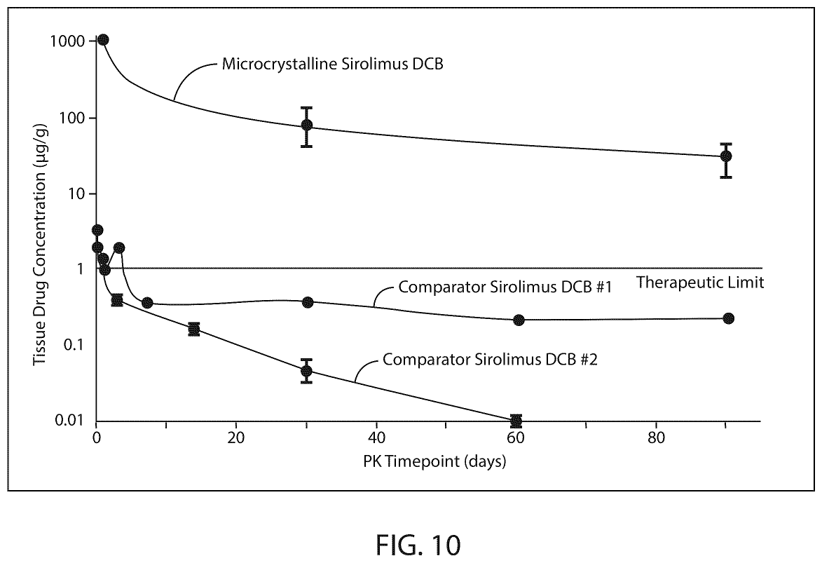

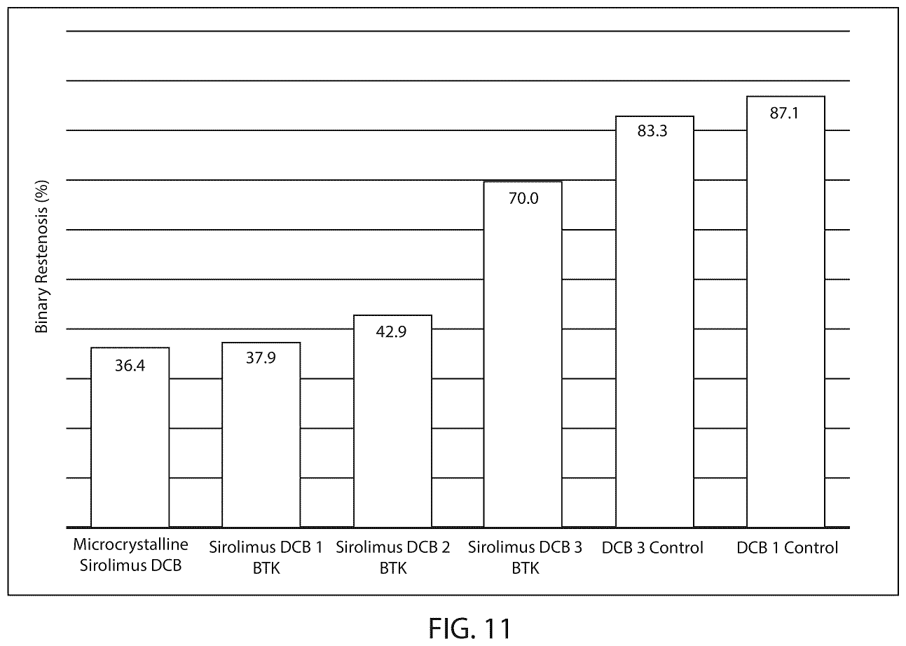

In some embodiments, the cationic agent 406 can be disposed or dispersed within areas between crystals 404, such as at touchpoints between crystals 404. In this configuration, the cationic agent 406 can also partially or fully coat the crystals 404. Referring now to FIG. 5, a schematic cross-sectional view is shown of a coating in accordance with various embodiments herein. FIG. 5 is generally similar to FIG. 4. However, in this view, the cationic agent 406 is shown as being disposed at touchpoints (or narrow areas) between the crystals 404. In this configuration, portions of the crystals 404 are uncoated with cationic agent 406. Exposed or “under-wetted” crystals 404 can have a biological effect. As larger portions of the crystals 404 are uncovered by the cationic agent 406, the exposed or uncoated portion of the crystals 404 can react with the blood and induce fibrin formation after being transferred to the vessel wall. Specifically, sirolimus crystals 404 can form a fibrin layer over sirolimus crystals transferred to the vessel wall to form a drug depot resistant to being washed away or dissolved. As shown in FIG. 10, microcrystalline sirolimus crystals 404 delivered with a cationic agent 406 according to preparations described herein, remained concentrated in the tissue at above therapeutic doses for at least 90 days. In comparison, sirolimus delivered by other commercial drug coatings (comparator sirolimus DCB #1, comparator sirolimus DCB #2), that do not use cationic agent or microcrystalline sirolimus crystals, failed to deliver the active agent in a manner that maintained therapeutic drug concentrations for a prolonger time period. As shown in FIG. 11, the prolonged effective therapeutic concentration of sirolimus provided by the microcrystalline form and cationic agent excipient resulted in less incidence of restenosis when compared to other commercial drug coatings (sirolimus DCB #1 BTK, sirolimus DCB #2 BTK, sirolimus DCB #3 BTK) and control testing (DCB 3 control, DCB 1 control). Placing cationic agent 406 at touchpoints between the crystals 404 can also leave a number of narrow gaps 502 remaining in the active agent layer 206. These narrow gaps can extend from the exterior of the active agent layer 206 partially through of the active agent layer 206 or entirely through the active agent layer 206 to form open or bare spots on the intermediate layer 402. In some embodiments, these gaps 502 can remain empty in the final coating. In other embodiments, these gaps 502 can be filled with another substance. As shown in FIGS. 6-7, in some embodiments, the active agent crystals 404 can be non-uniformly deposited on the substrate 202 or the intermediate layer 402 forming a porous structure having a plurality of pores 502. In this configuration, the cationic agent 406 can be positioned at least at the touch points between the active agent crystals 404 and active agent crystals 404 and the substrate 202 or the intermediate layer 402. These pores can extend from the exterior of the active agent layer 206 partially through of the active agent layer 206 or entirely through the active agent layer 206 to form open or bare spots on the intermediate layer 402.

In some embodiments, the cationic agent 406 and active agent crystals 404 can comprise distinct layers. In this configuration, the active agent crystals 404 form a sublayer on the substrate 202 or the intermediate layer 402 wherein the cationic agent 406 is deposited over the active agent crystal 404 sublayer. The cationic agent 406 can partially or fully coat the active agent crystals 404 sublayer.

Referring now to FIG. 6, a schematic cross-sectional view is shown of a portion of an active agent layer 206 of a coating in accordance with various embodiments herein. As before, the active agent layer 206 can include a plurality of crystals 404, a cationic agent 406, and gaps 502. As shown in FIG. 5, the cationic agent 406 can act as a binder to adhere adjacent crystals 404 together. The adhered crystals 404 can form a coating of uniform thickness on the surface of the intermediate layer 402. In some embodiments, the adhered crystals 404 can form crystal aggregates on the intermediate layer 402 or porous active agent layer 206 with gaps or pores 502 in the active agent layer 206. The aggregate crystals 404 of porous active agent layer 206 can comprise uniform or uneven coating thickness. In some embodiments, the gaps 502 can take up about 5, 10, 15, 20, 25, 30, or 35 percent or more of the active agent layer 206 by volume, or an amount falling within a range between any of the foregoing.

Various conditions during coating processes and/or storage of the medical device after coating can be controlled to enhance properties of the coating on the medical device. For example, in various embodiments the temperature and/or the humidity during the coating process can be controlled. In various embodiments, the humidity can be greater than or equal to 20, 22.5, 25, 27.5, 30, 32.5, 35, or 37.5 percent relative humidity. In various embodiments, the humidity can be less than or equal to 40, 37.5, 35, 32.5, 30, 27.5, or 25 percent relative humidity. In various embodiments, the relative humidity can be fall within a range between any of the foregoing. In various embodiments, the temperature can be at approximately standard room temperature. In some embodiments, the temperature can be approximately 18, 19, 20, 21, 22, 23, or 24 degrees Celsius, or a temperature falling within a range between any of the foregoing.

### Active Agents

In various embodiments herein, the active agent is in a crystalline form. By way of example, in various embodiments, the active agent can be at least 50, 60, 70, 80, 90, 95, 98, 99, or 100 percent by wt. crystalline, or an amount falling within a range between any of the foregoing. Conversely, the amount of the active agent in an amorphous form can be less than 50, 40, 30, 20, 10, 5, 2, or 1 percent by wt. of the total amount of active agent, or an amount falling within a range between any of the foregoing.

Without being bound by theory, the size of the crystals as well as the zeta potential of the same impacts the amount of active agent transfer to the targeted tissue as well as retention of the active agent by the targeted tissue.

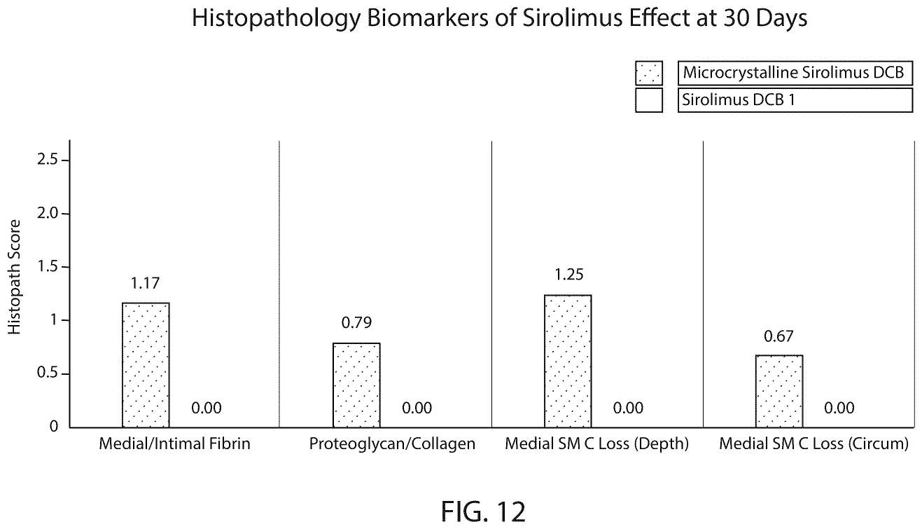

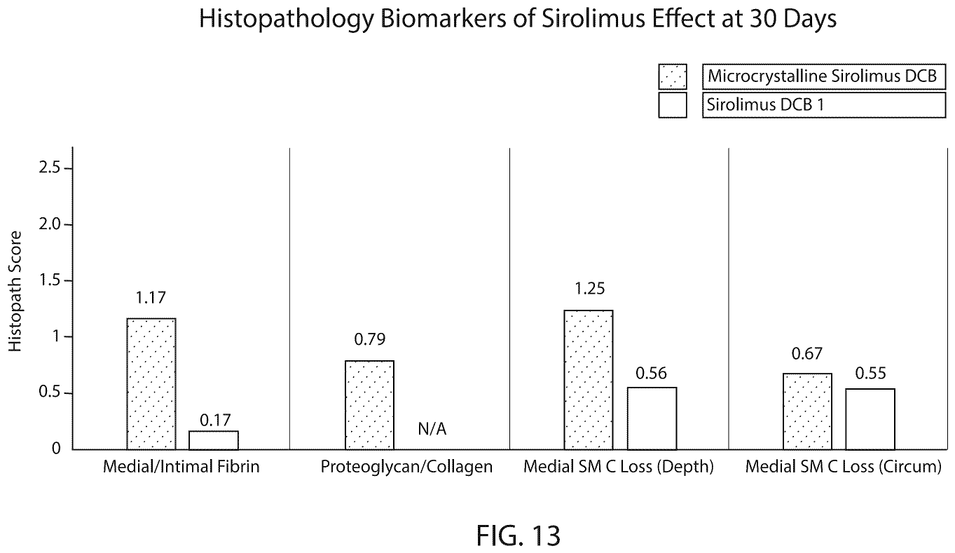

The active agent used herein can be in the form of a crystalline material with very small average crystal sizes. In various embodiments, the microcrystalline active agent is at least 90 percent crystalline or 95 percent crystalline. In various embodiments, the amount of amorphous active agent in the active agent composition is less than 10 percent or less than 5 percent by weight. The high crystallinity of the active agent improves the transfer of the active agent from the delivery device to the tissue wall. In various embodiments, the average size of crystals (number average) can be less than 100, 50, 40, 30, 25, 20, 15, or 10 micrometers, or an average size falling within a range between any of the foregoing. The particle size of the microcrystalline active agent, in combination with a cationic agent, aids in the transfer of the microcrystalline active agent to the tissue wall and improves wall adherence for more prolonged absorption into the tissue wall. As shown in FIGS. 12 and 13, microcrystalline active agent (Microcrystalline Sirolimus DCB) delivered with a cationic agent demonstrated prolonged therapeutic effects up to 30 days after initial treatment. In comparison, the same active agent delivered from a commercial drug delivery coating (Sirolimus DCB 1) failed to little effect 30 days after delivery.

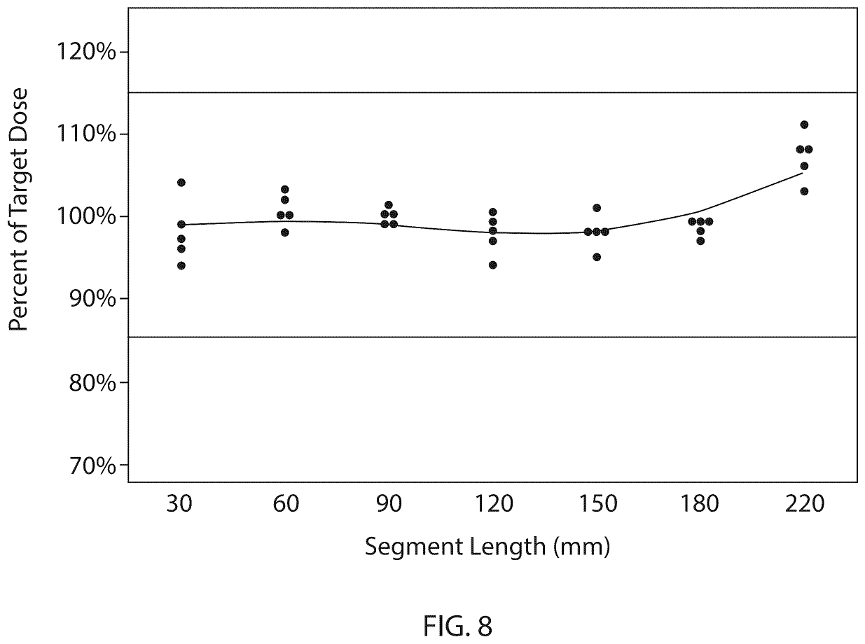

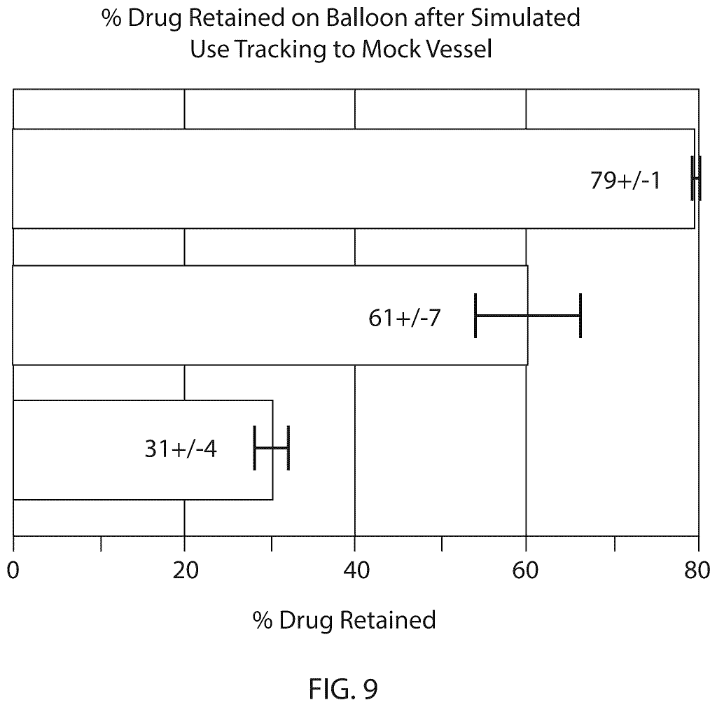

The zeta potential of the active agent crystals can vary. In some embodiments, the zeta potential can be greater than or equal to −60 mV, −57 mV, −54 mV, −52 mV, −49 mV, or −46 mV. In some embodiments, the zeta potential can be less than or equal to −35 mV, −37 mV, −39 mV, −42 mV, −44 mV, or −46 mV. In some embodiments, the zeta potential can fall within a range of −60 mV to −35 mV, or −57 mV to −37 mV, or −54 mV to −39 mV, or −50 mV to −42 mV. Fully or partially coating the microcrystalline active agent crystals with the cationic agent induces a positive aggregate zeta potential, which improves the deliverability of the microcrystalline active agent to the tissue wall and the adherence of the microcrystalline active agent to the tissue wall. The cationic agent can provide a positive zeta potential with a partial coating or full encapsulation of the microcrystalline active agent crystals. As shown in FIG. 9, the positive zeta potential of the microcrystalline active agent provided by the cationic agent improves adhesion of the active agent to the substrate 202 or intermediate layer 402 when the drug delivery device is tracked through a simulated vessel thereby minimizing loss during tracking of the drug delivery device to the treatment site. In comparison, drug delivery coatings that deliver active agent without a cationic agent (sirolimus DCB 1 and sirolimus DCB 2) lost considerable active agent during tracking of the drug delivery device to the treatment site. Likewise, the positive zeta potential imparted by the cationic agent improves transfer of the active agent to the treatment site when the drug delivery device is expanded at the treatment site. In particular, the positive zeta potential imparted by the cationic agent with a hydrophilic basecoat provides consistent active agent delivery along the length of the drug delivery device as shown in FIG. 8.

It will be appreciated that active agents of embodiments herein can include agents having many different types of activities. In various embodiments herein, active agents can specifically include hydrophobic active agents. The terms “active agent” and “therapeutic agent” as used herein shall be coterminous unless the context dictates otherwise. Hydrophobic active agents can specifically include those having solubility in water of less than about 100 μg/mL at 25 degrees Celsius and neutral pH. In various embodiments, hydrophobic active agents can specifically include those having solubility in water of less than about 10 μg/mL at 25 degrees Celsius and neutral pH. In some embodiments, hydrophobic active agents can specifically include those having solubility in water of less than about 5 μg/ml at 25 degrees Celsius and neutral pH.

In some exemplary embodiments, active agents can include, but are not limited to, antiproliferatives such as paclitaxel and limus drugs such as sirolimus (rapamycin), rapalogs, zotarolimus, everolimus, temsirolimus, pimecrolimus, tacrolimus, and ridaforolimus; analgesics and anti-inflammatory agents such as aloxiprin, auranofin, azapropazone, benorylate, diflunisal, etodolac, fenbufen, fenoprofen calcim, flurbiprofen, ibuprofen, indomethacin, ketoprofen, meclofenamic acid, mefenamic acid, nabumetone, naproxen, oxyphenbutazone, phenylbutazone, piroxicam, sulindac; anti-arrhythmic agents such as amiodarone HCl, disopyramide, flecainide acetate, quinidine sulphate; anti-bacterial agents such as benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim; anti-coagulants such as dicoumarol, dipyridamole, nicoumalone, phenindione; anti-hypertensive agents such as amlodipine, benidipine, darodipine, dilitazem HCl, diazoxide, felodipine, guanabenz acetate, isradipine, minoxidil, nicardipine HCl, nifedipine, nimodipine, phenoxybenzamine HCl, prazosin HCL, reserpine, terazosin HCL; anti-muscarinic agents: atropine, benzhexol HCl, biperiden, ethopropazine HCl, hyoscyamine, mepenzolate bromide, oxyphencylcimine HCl, tropicamide; anti-neoplastic agents and immunosuppressants such as aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine HCl, tamoxifen citrate, testolactone; beta-blockers such as acebutolol, alprenolol, atenolol, labetalol, metoprolol, nadolol, oxprenolol, pindolol, propranolol; cardiac inotropic agents such as amrinone, digitoxin, digoxin, enoximone, lanatoside C, medigoxin; corticosteroids such as beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone; lipid regulating agents such as bezafibrate, clofibrate, fenofibrate, gemfibrozil, probucol; nitrates and other anti-anginal agents such as amyl nitrate, glyceryl trinitrate, isosorbide dinitrate, isosorbide mononitrate, pentaerythritol tetranitrate; therapeutic peptides and proteins, and the like. Other exemplary embodiments of active agents include, but are not limited to, active agents for treatment of hypertension (HTN), such as guanethidine.

Active agents herein can include those used to prevent restenosis of a vessel, such as restenosis of an artery. In some embodiments herein, the active agent can be a macrocyclic lactone. Active agents herein can include sirolimus (rapamycin), various rapalogs, tacrolimus, everolimus, paclitaxel, biolimus, zotarolimus, pimecrolimus, and the like. In some embodiments, the active agent is specifically crystalline sirolimus.

In some embodiments, a hydrophobic active agent can be conjugated to a cationic agent. The conjugation can include a hydrophobic active agent covalently bonded to the cationic agent. In some embodiments wherein the hydrophobic agent is conjugated to the cationic agent a linking agent can be used to attach the hydrophobic agent to the cationic agent. Suitable linking agents include, but are not limited to, polyethylene glycol, polyethylene oxide and polypeptides of naturally-occurring and non-naturally occurring amino acids. In some embodiments, linking agents can be biodegradable or cleavable in vivo to assist in release of the hydrophobic active agents. Exemplary linking agents can further include alkane or aromatic compounds with heteroatom-substitutions such as N, S, Si, Se or O.

### Hydrophilic Base Coatings

Various embodiments herein can include a base coat disposed between the substrate and the layer including the active agent. In some embodiments, the base coat can include hydrophilic polymers. However, in some embodiments, the base coat can include hydrophobic polymers.

One class of hydrophilic polymers useful as polymeric materials for hydrophilic base coat formation is synthetic hydrophilic polymers. Synthetic hydrophilic polymers that are biostable (i.e., that show no appreciable degradation in vivo) can be prepared from any suitable monomer including acrylic monomers, vinyl monomers, ether monomers, or combinations of any one or more of these types of monomers. Acrylic monomers include, for example, methacrylate, methyl methacrylate, hydroxyethyl methacrylate, hydroxyethyl acrylate, methacrylic acid, acrylic acid, glycerol acrylate, glycerol methacrylate, acrylamide, methacrylamide, dimethylacrylamide (DMA), and derivatives and/or mixtures of any of these. Vinyl monomers include, for example, vinyl acetate, vinylpyrrolidone, vinyl alcohol, and derivatives of any of these. Ether monomers include, for example, ethylene oxide, propylene oxide, butylene oxide, and derivatives of any of these. Examples of polymers that can be formed from these monomers include poly(acrylamide), poly(methacrylamide), poly(vinylpyrrolidone), poly(acrylic acid), poly(ethylene glycol), poly(vinyl alcohol), and poly(HEMA). Examples of hydrophilic copolymers include, for example, methyl vinyl ether/maleic anhydride copolymers and vinyl pyrrolidone/(meth)acrylamide copolymers. Mixtures of homopolymers and/or copolymers can be used.

Examples of some acrylamide-based polymers, such as poly(N,N-dimethylacrylamide-co-aminopropylmethacrylamide) and poly(acrylamide-co-N,N-dimethylaminopropylmethacrylamide) are described in example 2 of U.S. Pat. No. 7,807,750 (Taton et al.), the disclosure of which is incorporated herein by reference.

Other hydrophilic polymers that can be useful in the present disclosure are derivatives of acrylamide polymers with photoreactive groups. One such representative hydrophilic polymer can be the copolymerization of N-[3-(4-benzoylbenzamido)propyl]methacrylamide (Formula I) with N-(3-aminopropyl)methacrylamide (Formula II) to produce the polymer poly[N-3-aminopropyl)methacrylamide-co-N-[3-(4-benzoylbenzamido)propyl]methacrylamide (Formula III). The preparation of the polymer is disclosed in Example 1 of U.S. Pat. No. 11,147,902 (to Lodhi, et al.), the full content of which is incorporated herein by reference.

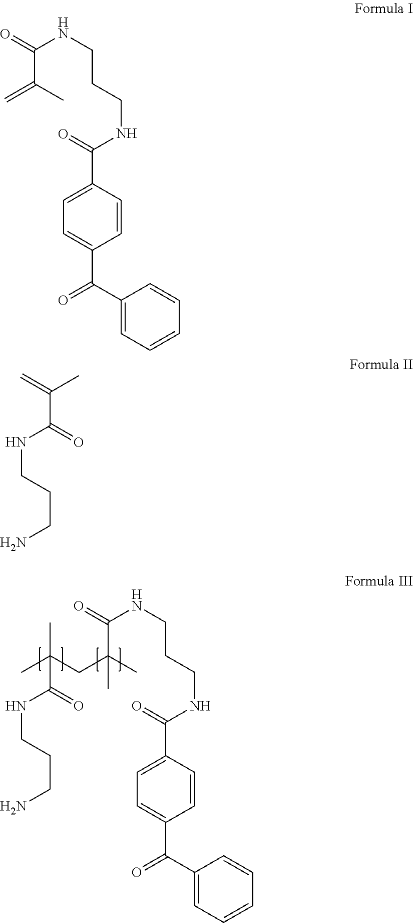

In some embodiments, the hydrophilic polymer can be a vinyl pyrrolidone polymer, or a vinyl pyrrolidone/(meth)acrylamide copolymer such as poly(vinylpyrrolidone-co-methacrylamide). If a PVP copolymer is used, it can be a copolymer of vinylpyrrolidone and a monomer selected from the group of acrylamide monomers. Exemplary acrylamide monomers include (meth)acrylamide and (meth)acrylamide derivatives, such as alkyl(meth)acrylamide, as exemplified by dimethylacrylamide, and aminoalkyl(meth)acrylamide, as exemplified by aminopropylmethacrylamide and dimethylaminopropylmethacrylamide. For example, poly(vinylpyrrolidone-co-N,N dimethylaminopropylmethacrylamide) is described in example 2 of U.S. Pat. No. 7,807,750 (Taton et al.).

In one embodiment, the polymers and copolymers as described are derivatized with one or more photoactivatable group(s). Exemplary photoreactive groups that can be pendent from biostable hydrophilic polymer include aryl ketones, such as acetophenone, benzophenone, anthraquinone, anthrone, quinone, and anthrone-like heterocycles. Aryl ketones herein can specifically include diaryl ketones. Polymers herein can provide a hydrophilic polymer having a pendent activatable photogroup that can be applied to the expandable and collapsible structure, and can then treated with actinic radiation sufficient to activate the photogroups and cause covalent bonding to a target, such as the material of the expandable and collapsible structure. Use of photo-hydrophilic polymers can be used to provide a durable coating of a flexible hydrogel matrix, with the hydrophilic polymeric materials covalently bonded to the material of the expandable and collapsible structure.

A hydrophilic polymer having pendent photoreactive groups can be used to prepare the flexible hydrogel coating. Methods of preparing hydrophilic polymers having photoreactive groups are known in the art. For example, methods for the preparation of photo-PVP are described in U.S. Pat. No. 5,414,075, the disclosure of which is incorporated herein by reference. Hydrophilic photo-polyacrylamide polymers such as poly(acrylamide-co-N-(3-(4-benzoylbenzamido)propyl)methacylamide), “Photo-PAA”, and derivatives thereof can be used to form hydrophilic base coats in exemplary embodiments of the present disclosure. Methods for the preparation of photo-polyacrylamide are described in U.S. Pat. No. 6,007,833, the disclosure of which is incorporated herein by reference.

Other embodiments of hydrophilic base coats include derivatives of photo-polyacrylamide polymers incorporating additional reactive moieties. Some exemplary reactive moieties include N-oxysuccinimide and glycidyl methacrylate. Representative photo-polyacrylamide derivatives incorporating additional reactive moieties include poly(acrylamide-co-maleic-6-aminocaproic acid-N-oxysuccinimide-co-N-(3-(4-benzoylbenzamido)propyl)methacrylamide) and poly(acrylamide-co-(3-(4-benzoylbenzamido)propyl)methacrylamide)-co-glycidylmethacrylate. Additional photo-polyacrylamide polymers incorporating reactive moieties are described in U.S. Pat. No. 6,465,178 (to Chappa, et al.), U.S. Pat. No. 6,762,019 (to Swan, et al.) and U.S. Pat. No. 7,309,593 (to Ofstead, et al.), the disclosures of which are herein incorporated by reference.

Other embodiments of exemplary hydrophilic base coats that include derivatives of photo-polyacrylamide polymers incorporating additional reactive moieties can be found in U.S. Pat. No. 6,514,734 (to Clapper, et al.), the disclosure of which is incorporated herein by reference in its entirety.

In yet other embodiments, the hydrophilic base coat can include derivatives of photo-polyacrylamide polymers incorporating charged moieties. Charged moieties include both positively and negatively charged species. Exemplary charged species include, but are not limited to, sulfonates, phosphates and quaternary amine derivatives. Some examples include the negatively charged species N-acetylated poly(acrylamide-co-sodium-2-acrylamido-2-methylpropanesulfonate-co-N-(3-(4-benzoylbenzamido)propyl)methacrylamide)-co-methoxy poly(ethylene glycol) monomethacrylate. Other negatively charged species that can be incorporated into the hydrophilic base coat are described in U.S. Pat. No. 4,973,993, the disclosure of which is incorporated herein by reference in its entirety. Positively charged species can include poly(acrylamide-co-N-(3-(4-benzoylbenzamido)propyl)methacrylamide)-co-(3-(methacryloylamino)propyl)trimethylammonium chloride. Other positively charged species that can be incorporated into the hydrophilic base coat are described in U.S. Pat. No. 5,858,653 (to Duran et al.), the disclosure of which is incorporated herein by reference in its entirety.

In another embodiment, the polymers and copolymers as described are derivatized with one or more polymerizable group(s). Polymers with pendent polymerizable groups are commonly referred to as macromers. The polymerizable group(s) can be present at the terminal portions (ends) of the polymeric strand or can be present along the length of the polymer. In one embodiment polymerizable groups are located randomly along the length of the polymer.

Exemplary hydrophilic polymer coatings can be prepared using polymer grafting techniques. Polymer grafting techniques can include applying a nonpolymeric grafting agent and monomers to a substrate surface then causing polymerization of the monomers on the substrate surface upon appropriate activation (for example, but not limited to, UV radiation) of the grafting agent. Grafting methods producing hydrophilic polymeric surfaces are exemplified in U.S. Pat. Nos. 7,348,055; 7,736,689 and 8,039,524 (all to Chappa et al.) the full disclosures of which are incorporated herein by reference.

### Cross-Linking Agents

In various embodiments, the coating can include one or more crosslinking agents in one or more layers of the coating. A crosslinking agent can promote the association of polymers in the coating, or the bonding of polymers to the coated surface. The choice of a particular crosslinking agent can depend on the ingredients of the coating composition.

Suitable crosslinking agents can include two or more activatable groups, which can react with the polymers in the composition. Suitable activatable groups can include photoreactive groups as described herein, like aryl ketones, such as acetophenone, benzophenone, anthraquinone, anthrone, quinone, and anthrone-like heterocycles. A crosslinking agent including a photoreactive group can be referred to as a photo-crosslinker or photoactivatable crosslinking agent. The photoactivatable crosslinking agent can be ionic, and can have good solubility in an aqueous composition. Thus, in some embodiments, at least one ionic photoactivatable crosslinking agent can be used to form the coating. The ionic crosslinking agent can include an acidic group or salt thereof, such as selected from sulfonic acids, carboxylic acids, phosphonic acids, salts thereof, and the like. Exemplary counter ions include alkali, alkaline earths metals, ammonium, protonated amines, and the like.

Exemplary ionic photoactivatable crosslinking agents include 4,5-bis(4-benzoylphenylmethyleneoxy) benzene-1,3-disulfonic acid or salt; 2,5-bis(4-benzoylphenylmethyleneoxy)benzene-1,4-disulfonic acid or salt; 2,5-bis(4-benzoylmethyleneoxy)benzene-1-sulfonic acid or salt; N,N-bi s[2-(4-benzoylbenzyloxy)ethyl]-2-aminoethanesulfonic acid or salt, and the like. See U.S. Pat. No. 6,077,698 (Swan et al.), U.S. Pat. No. 6,278,018 (Swan), U.S. Pat. No. 6,603,040 (Swan) and U.S. Pat. No. 7,138,541 (Swan) the disclosures of which are incorporated herein by reference.

Other exemplary ionic photoactivatable crosslinking agents include ethylenebis(4-benzoylbenzyldimethylammonium) dibromide and hexamethylenebis(4-benzoylbenzyldimethylammonium) dibromide and the like. See U.S. Pat. No. 5,714,360 (Swan et al.) the disclosures of which are incorporated herein by reference. In yet other embodiments, restrained multifunctional reagents with photoactivable crosslinking groups can be used. In some examples these restrained multifunctional reagents include tetrakis (4-benzoylbenzyl ether) of pentaerthyritol and the tetrakis (4-benzoylbenzoate ester) of pentaerthyritol. See U.S. Pat. No. 5,414,075 (Swan et al.) and U.S. Pat. No. 5,637,460 (Swan et al.) the disclosures of which are incorporated herein by reference.

Additional crosslinking agents can include those having formula Photo¹-LG-Photo², wherein Photo¹ and Photo² independently represent at least one photoreactive group and LG represents a linking group comprising at least one silicon or at least one phosphorus atom, wherein the degradable linking agent comprises a covalent linkage between at least one photoreactive group and the linking group, wherein the covalent linkage between at least one photoreactive group and the linking group is interrupted by at least one heteroatom. See U.S. Pat. No. 8,889,760 (Kurdyumov, et al.), the disclosure of which is incorporated herein by reference. Further crosslinking agents can include those having a core molecule with one or more charged groups and one or more photoreactive groups covalently attached to the core molecule by one or more degradable linkers. See U.S. Publ. Pat. App. No. 2011/0144373 (Swan, et al.), the disclosure of which is incorporated herein by reference.

Crosslinking agents used in accordance with embodiments herein can include those with at least two photoreactive groups. Exemplary crosslinking agents are described in U.S. Pat. No. 8,889,760, the content of which is herein incorporated by reference in its entirety.

In some embodiments, crosslinking agents herein can have a molecular weight of less than about 1500 kDa. In some embodiments the crosslinking agent can have a molecular weight of less than about 1200, 1100, 1000, 900, 800, 700, 600, 500, or 400.

In some embodiments, at least one of the first and second crosslinking agents comprising a linking agent having formula Photo¹-LG-Photo², wherein Photo¹ and Photo², independently represent at least one photoreactive group and LG represents a linking group comprising at least one silicon or at least one phosphorus atom, there is a covalent linkage between at least one photoreactive group and the linking group, wherein the covalent linkage between at least one photoreactive group and the linking group is interrupted by at least one heteroatom.

In some embodiments, at least one of the first and second crosslinking agents comprising a linking agent having a formula selected from:
(a)

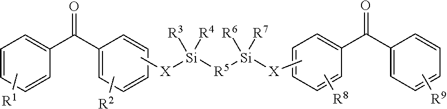

wherein R¹, R², R⁸ and R⁹ are any substitution; R³, R⁴, R⁶ and R⁷ are alkyl, aryl, or a combination thereof; R⁵ is any substitution; and each X, independently, is O, N, Se, S, or alkyl, or a combination thereof;
(b)

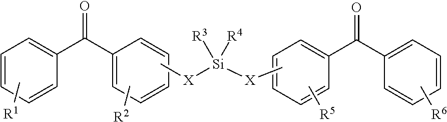

wherein R¹ and R⁵ are any substitution; R² and R⁴ can be any substitution, except OH; R³ can be alkyl, aryl, or a combination thereof; and X, independently, are O, N, Se, S, alkylene, or a combination thereof;
(c)

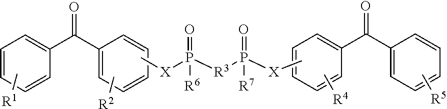

wherein R¹, R², R⁴ and R⁵ are any substitution; R³ is any substitution; R⁶ and R⁷ are alkyl, aryl, or a combination thereof; and each X can independently be O, N, Se, S, alkylene, or a combination thereof; and
(d)

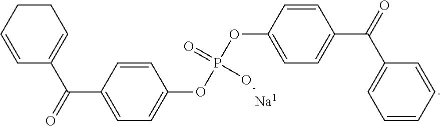 [SMILES: O=C(C1=CCCC=C1)c1ccc(OP(=O)(O)Oc2ccc(C(=O)c3ccccc3)cc2)cc1]

In a particular embodiment, the crosslinking agent can be bis(4-benzoylphenyl) phosphate. In some embodiments, the photoactivatable crosslinking agent can be ionic, and can have good solubility in an aqueous composition, such as the first and/or second coating composition. Thus, in some embodiments, at least one ionic photoactivatable crosslinking agent is used to form the coating. In some cases, an ionic photoactivatable crosslinking agent can crosslink the polymers within the second coating layer which can also improve the durability of the coating.

Any suitable ionic photoactivatable crosslinking agent can be used. In some embodiments, the ionic photoactivatable crosslinking agent is a compound of formula I: X1-Y—X2 where Y is a radical containing at least one acidic group, basic group, or a salt of an acidic group or basic group. X1 and X2 are each independently a radical containing a latent photoreactive group. The photoreactive groups can be the same as those described herein. Spacers can also be part of X1 or X2 along with the latent photoreactive group. In some embodiments, the latent photoreactive group includes an aryl ketone or a quinone.

The radical Y in formula I provides the desired water solubility for the ionic photoactivatable crosslinking agent. The water solubility (at room temperature and optimal pH) is at least about 0.05 mg/ml. In some embodiments, the solubility is about 0.1 to about 10 mg/ml or about 1 to about 5 mg/ml.

In some embodiments of formula I, Y is a radical containing at least one acidic group or salt thereof. Such a photoactivatable crosslinking agent can be anionic depending upon the pH of the coating composition. Suitable acidic groups include, for example, sulfonic acids, carboxylic acids, phosphonic acids, and the like. Suitable salts of such groups include, for example, sulfonate, carboxylate, and phosphate salts. In some embodiments, the ionic crosslinking agent includes a sulfonic acid or sulfonate group.

Suitable counter ions include alkali, alkaline earths metals, ammonium, protonated amines, and the like.

For example, a compound of formula I can have a radical Y that contains a sulfonic acid or sulfonate group; X1 and X2 can contain photoreactive groups such as aryl ketones. Such compounds include 4,5-bis(4-benzoylphenylmethyleneoxy)benzene-1,3-disulfonic acid or salt; 2,5-bis(4-benzoylphenylmethyleneoxy)benzene-1,4-disulfonic acid or salt; 2,5-bis(4-benzoylmethyleneoxy)benzene-1-sulfonic acid or salt; N,N-bis[2-(4-benzoylbenzyloxy)ethyl]-2-aminoethanesulfonic acid or salt, and the like. See U.S. Pat. No. 6,278,018. The counter ion of the salt can be, for example, ammonium or an alkali metal such as sodium, potassium, or lithium.

In other embodiments of formula I, Y can be a radical that contains a basic group or a salt thereof. Such Y radicals can include, for example, an ammonium, a phosphonium, or a sulfonium group. The group can be neutral or positively charged, depending upon the pH of the coating composition. In some embodiments, the radical Y includes an ammonium group. Suitable counter ions include, for example, carboxylates, halides, sulfate, and phosphate. For example, compounds of formula I can have a Y radical that contains an ammonium group; X1 and X2 can contain photoreactive groups that include aryl ketones. Such photoactivatable crosslinking agents include ethylenebis(4-benzoylbenzyldimethylammonium) salt; hexamethylenebis (4-benzoylbenzyldimethylammonium) salt; 1,4-bis(4-benzoylbenzyl)-1,4-dimethylpiperazinediium) salt, bis(4-benzoylbenzyl)hexamethylenetetraminediium salt, bi s[2-(4-benzoylbenzyldimethylammonio)ethyl]-4-benzoylbenzylmethylammonium salt; 4,4-bis(4-benzoylbenzyl)morpholinium salt; ethylenebis[(2-(4-benzoylbenzyldimethylammonio)ethyl)-4-benzoylbenzylmethylammonium] salt; and 1,1,4,4-tetrakis(4-benzoylbenzyl)piperzinediium salt. See U.S. Pat. No. 5,714,360. The counter ion is typically a carboxylate ion or a halide. On one embodiment, the halide is bromide.

In other embodiments, the ionic photoactivatable crosslinking agent can be a compound having the formula:

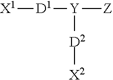

wherein X¹ includes a first photoreactive group; X² includes a second photoreactive group; Y includes a core molecule; Z includes at least one charged group; D¹ includes a first degradable linker; and D² includes a second degradable linker. Additional exemplary degradable ionic photoactivatable crosslinking agents are described in US Patent Application Publication US 2011/0144373 (Swan et al., “Water Soluble Degradable Crosslinker”), the disclosure of which is incorporated herein by reference.

In some aspects a non-ionic photoactivatable crosslinking agent can be used. In one embodiment, the non-ionic photoactivatable crosslinking agent has the formula XR¹R²R³R⁴ where X is a chemical backbone, and R¹, R², R³, and R⁴ are radicals that include a latent photoreactive group. Exemplary non-ionic crosslinking agents are described, for example, in U.S. Pat. Nos. 5,414,075 and 5,637,460 (Swan et al., “Restrained Multifunctional Reagent for Surface Modification”). Chemically, the first and second photoreactive groups, and respective spacers, can be the same or different.

In other embodiments, the non-ionic photoactivatable crosslinking agent can be represented by the formula:

PG²-LE²-X-LE¹-PG¹

wherein PG¹ and PG² include, independently, one or more photoreactive groups, for example, an aryl ketone photoreactive group, including, but not limited to, aryl ketones such as acetophenone, benzophenone, anthraquinone, anthrone, anthrone-like heterocycles, their substituted derivatives or a combination thereof; LE¹ and LE² are, independently, linking elements, including, for example, segments that include urea, carbamate, or a combination thereof; and X represents a core molecule, which can be either polymeric or non-polymeric, including, but not limited to a hydrocarbon, including a hydrocarbon that is linear, branched, cyclic, or a combination thereof aromatic, non-aromatic, or a combination thereof; monocyclic, polycyclic, carbocyclic, heterocyclic, or a combination thereof benzene or a derivative thereof; or a combination thereof. Other non-ionic crosslinking agents are described, for example, in U.S. application Ser. No. 13/316,030 filed Dec. 9, 2011 (Publ. No. US 2012/0149934) (Kurdyumov, “Photocrosslinker”), the disclosure of which is incorporated herein by reference.

Further embodiments of non-ionic photoactivatable crosslinking agents can include, for example, those described in US Pat. Publication 2013/0143056 (Swan et al., “Photo-Vinyl Primers/Crosslinkers”), the disclosure of which is incorporated herein by reference. Exemplary crosslinking agents can include non-ionic photoactivatable crosslinking agents having the general formula R¹— X— R², wherein R¹ is a radical comprising a vinyl group, X is a radical comprising from about one to about twenty carbon atoms, and R² is a radical comprising a photoreactive group.

A single photoactivatable crosslinking agent or any combination of photoactivatable crosslinking agents can be used in forming the coating. In some embodiments, at least one nonionic crosslinking agent such as tetrakis(4-benzoylbenzyl ether) of pentaerythritol can be used with at least one ionic crosslinking agent. For example, at least one non-ionic photoactivatable crosslinking agent can be used with at least one cationic photoactivatable crosslinking agent such as an ethylenebis(4-benzoylbenzyldimethylammonium) salt or at least one anionic photoactivatable crosslinking agent such as 4,5-bis(4-benzoyl-phenylmethyleneoxy)benzene-1,3-disulfonic acid or salt. In another example, at least one nonionic crosslinking agent can be used with at least one cationic crosslinking agent and at least one anionic crosslinking agent. In yet another example, a least one cationic crosslinking agent can be used with at least one anionic crosslinking agent but without a non-ionic crosslinking agent.

An exemplary crosslinking agent is disodium 4,5-bis[(4-benzoylbenzyl)oxy]-1,3-benzenedisulfonate (DBDS). This reagent can be prepared by combining 4,5-Dihydroxylbenzyl-1,3-disulfonate (CHBDS) with 4-bromomethylbenzophenone (BMBP) in THF and sodium hydroxide, then refluxing and cooling the mixture followed by purification and recrystallization (also as described in U.S. Pat. No. 5,714,360, incorporated herein by reference).

Further crosslinking agents can include the crosslinking agents described in U.S. Publ. Pat. App. No. 2010/0274012 (to Guire et al.) and U.S. Pat. No. 7,772,393 (to Guire et al.) the content of all of which is herein incorporated by reference.

In some embodiments, crosslinking agents can include boron-containing linking agents including, but not limited to, the boron-containing linking agents disclosed in US Pat. Publication 2013/0302529 entitled “Boron-Containing Linking Agents” by Kurdyumov et al., the content of which is herein incorporated by reference. By way of example, linking agents can include borate, borazine, or boronate groups and coatings and devices that incorporate such linking agents, along with related methods. In an embodiment, the linking agent includes a compound having the structure (I):

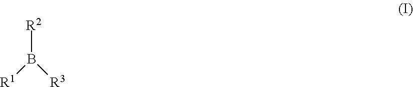

wherein R¹ is a radical comprising a photoreactive group; R² is selected from OH and a radical comprising a photoreactive group, an alkyl group and an aryl group; and R³ is selected from OH and a radical comprising a photoreactive group. In some embodiments the bonds B—R′, B—R² and B—R³ can be chosen independently to be interrupted by a heteroatom, such as O, N, S, or mixtures thereof.

Additional agents for use with embodiments herein can include stilbene-based reactive compounds including, but not limited to, those disclosed in U.S. Pat. No. 8,487,137, entitled “Stilbene-Based Reactive Compounds, Polymeric Matrices Formed Therefrom, and Articles Visualizable by Fluorescence” by Kurdyumov et al., the content of which is herein incorporated by reference.

Additional photoreactive agents, crosslinking agents, hydrophilic coatings, and associated reagents are disclosed in U.S. Pat. No. 8,513,320 (to Rooijmans et al.); U.S. Pat. No. 8,809,411 (to Rooijmans); and 2010/0198168 (to Rooijmans), the content of all of which is herein incorporated by reference.

Natural polymers can also be used to form the hydrophilic base coat. Natural polymers include polysaccharides, for example, polydextrans, carboxymethylcellulose, and hydroxymethylcellulose; glycosaminoglycans, for example, hyaluronic acid; polypeptides, for example, soluble proteins such as collagen, albumin, and avidin; and combinations of these natural polymers. Combinations of natural and synthetic polymers can also be used.

In some instances a tie layer can be used to form the hydrophilic base layer. In yet other instances the tie layer can be added to the hydrophilic base layer. The tie layer can act to increase the adhesion of the hydrophilic base layer to the substrate. In other embodiments, the tie layer can act to increase adhesion of the hydrophobic active agent to the hydrophilic base layer. Exemplary ties layers include, but are not limited to silane, butadiene, polyurethane and parylene. Silane tie layers are described in US Patent Publication 2012/0148852 (to Jelle, et al.), the content of which is herein incorporated by reference.

In exemplary embodiments, the hydrophilic base layer can include tannic acid, polydopamine or other catechol containing materials.

### Cationic Agents

Cationic agents used in embodiments herein can include compounds containing a portion having a positive charge in aqueous solution at neutral pH along with a portion that can exhibit affinity for hydrophobic surfaces (such as hydrophobic or amphiphilic properties) and can therefore interface with hydrophobic active agents. In some embodiments, cationic agents used in embodiments herein can include those having the general formula X—Y, wherein X is a radical including a positively charged group in aqueous solution at neutral pH and Y is a radical exhibiting hydrophobic properties. In some embodiments, the cationic agent can include a hydrophilic head and a hydrophobic tail, along with one or more positively charged groups, typically in the area of the hydrophilic head.

Cationic agents of the present disclosure can include salts of cationic agents at various pH ranges, such as, but not limited to, halide salts, sulfate salts, carbonate salts, nitrate salts, phosphate salts, acetate salts and mixtures thereof.

Cationic agents can specifically include cationic lipids and net neutral lipids that have a cationic group (neutral lipids with cationic groups). Exemplary lipids can include, but are not limited to, 3β-[N—(N′,N′-dimethylaminoethane)-carbamoyl]cholesterol hydrochloride (DC-cholesterol); 1,2-dioleoyl-3-trimethylammonium-propane (DOTAP); dimethyldioctadecylammonium (DDAB); 1,2-dioleoyl-sn-glycero-3-ethylphosphocholine (EPC); 1,2-di-O-octadecenyl-3-trimethylammonium propane (DOTMA); 1,2-di-(9Z-octadecenoyl)-3-dimethylammonium-propane (DODAP); 1,2-dilinoleyloxy-3-dimethylaminopropane (DLinDMA) and derivatives thereof. Additional lipids can include, but are not limited to, 1,2-dioleoyl-sn-glycero-3-phosphoethanolamine (DOPE); cholesterol; 1,2-dioctadecanoyl-sn-glycero-3-phosphocholine (DSPC); 1,2-distearoyl-sn-glycero-3-phosphoethanolamine (DSPE). Other cationic agents can include mono- or polyaminoalkanes such as spermine and spermidine.

Cationic agents can specifically include cationic polymers. Cationic agents can also include polycation-containing cyclodextrin (for example, but not limited to, amino cyclodextrin and derivatives thereof), amino dextran, histones, protamines, cationized human serum albumin, aminopolysaccharides such as chitosan, peptides such as poly-L-lysine, poly-L-ornithine, and poly(4-hydroxy-L-proline ester, and polyamines such as polyethylenimine (PEI; available from Sigma Aldrich), polyallylamine, polypropylenimine, polyamidoamine dendrimers (PAMAM; available from Sigma Aldrich), cationic polyoxazoline and poly(beta-aminoesters). Cationic agents can also specifically include cationic lipidoids (as described by K. T. Love in the publication PNAS 107, 1864-1869 (2010)). Other exemplary cationic polymers include, but are not limited to, block copolymers such as PEG-PEI and PLGA-PEI copolymers. Other exemplary cationic agents include positively charged gelatin (for example, base-treated gelatin), and the family of aminated cucurbit[n]urils (wherein n=5, 6, 7, 8, 10). In some embodiments, an acid can be added to the polycationic cationic agent suspension wherein the counterion of the acid can modify the properties of the cationic agent. The acid can be selected to provide a counter-ion including, but not limited to sulfates, phosphates, and acetates.

In some embodiments, the cationic agent is cross-linked with a bio-degradable crosslinker to selectively gel or otherwise increase the viscosity or density of the cationic agent. The relative viscosity and density of the cationic agent can affect the controlled release of the active agent from the medical device. In certain embodiments, the crosslinker can be added to the cationic agent to the cationic agent and active agent suspension prior to coating the medical device. The crosslinker can comprise a bio-degradable crosslinker such as, but not limited to degradable dialdehydes, carboxylic acid, or combinations thereof.

In other embodiments of the present disclosure, cationic agents containing a portion having a positive charge in aqueous solutions at neutral pH include the following Compounds (A-I):

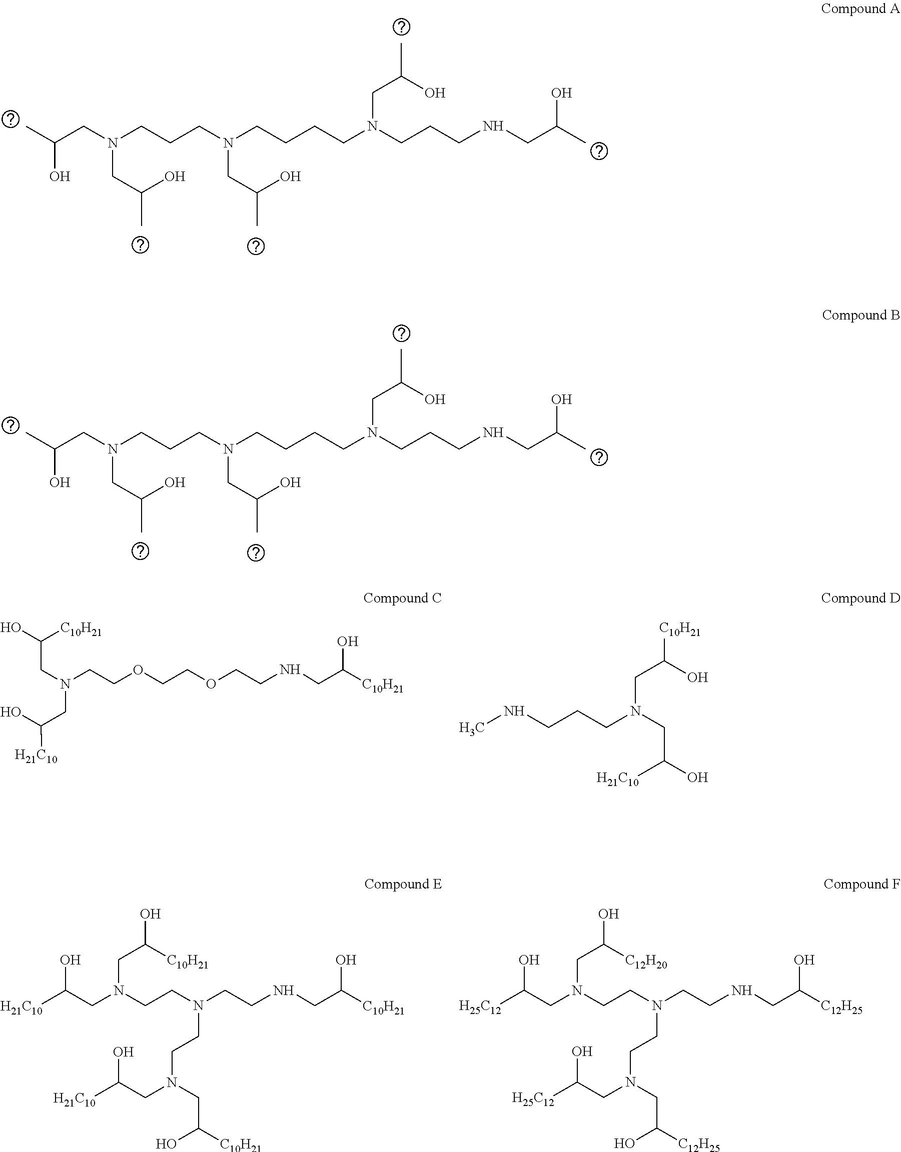

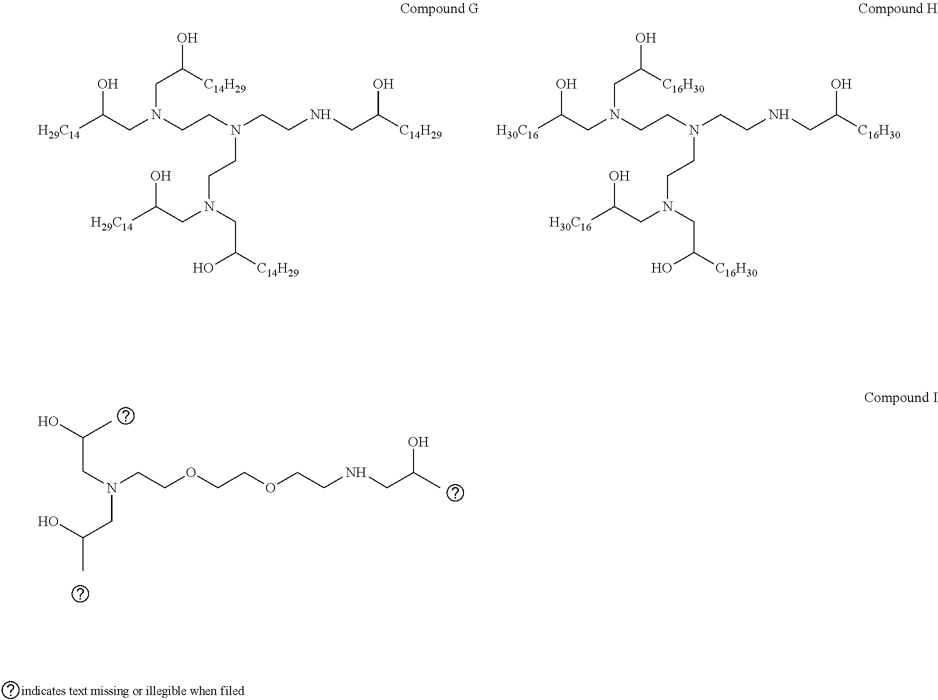

Additionally, other cationic agents include structures of the general Formula I:

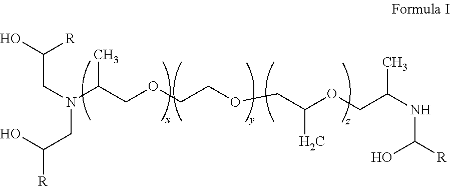

TABLE 1
Values for Variables x + z,
y and R for Compounds J-R of Formula I.

| Compound | x + z | y | R |
|---|---|---|---|
| Compound J | 6 | 12.5 | C₁₂H₂₅ |
| Compound K | 1.2 | 2 | C₁₂H₂₅ |
| Compound L | 6 | 39 | C₁₂H₂₅ |
| Compound M | 6 | 12.5 | C₁₄H₂₉ |
| Compound N | 1.2 | 2 | C₁₄H₂₉ |
| Compound O | 6 | 39 | C₁₄H₂₉ |
| Compound P | 6 | 12.5 | C₁₆H₃₃ |
| Compound Q | 1.2 | 2 | C₁₆H₃₃ |
| Compound R | 6 | 39 | C₁₆H₃₃ |

Cationic agents, such as those listed above, can generally be prepared by the reaction of an appropriate hydrophobic epoxide (e.g. oleyl epoxide) with a multi-functional amine (e.g. propylene diamine). Details of the synthesis of related cationic agents are described by K. T. Love in the publication PNAS 107, 1864-1869 (2010) and Ghonaim et al., Pharma Res 27, 17-29 (2010).

It will be appreciated that polyamide derivatives of PEI (PEI-amides) can also be applied as cationic agents. PEI-amides can generally be prepared by reacting PEI with an acid or acid derivative such as an acid chloride or an ester to form various PEI-amides. For example, PEI can be reacted with methyl oleate to form PEI-amides.

In yet other embodiments cationic agents can include moieties used to condense nucleic acids (for example lipids, peptides and other cationic polymers). In some instances these cationic agents can be used to form lipoplexes and polyplexes.

Exemplary embodiments of cationic agents can also include, but are not limited to, cationic agent derivatives that are photo reactive. Photo reactive groups are described below. Such cationic agent derivatives include PEI polymer derivatives of benzophenone and PAMAM polymer derivatives of benzophenone.

In some embodiments, the molecular weight of the cationic agent can be about 1.2 kDa, 2.5 kDa, 10 kDa, 25 kDa, 250 kDa or even, in some cases, 750 kDa. In yet other embodiments the molecular weight of the cationic agent can be in the range of 50-100 kDa, 70-100 kDa, 50-250 kDa, 25-100 kDa, 2.5-750 kDa or even, in some cases, 2.5-2,000 kDa. Other embodiments include molecular weights greater than 1.2 kDa, 2.5 kDa, 10 kDa, 25 kDa, 250 kDa or even, in some cases, greater than 750 kDa. Other embodiments can include cationic agents up to 2,000 kDa. Low molecular weight cationic agent monomers or low molecular weight cationic oligomers can be combined with hydrophobic active agent to produce a reactive coating. These reactive coatings can then be coated onto a substrate and thermally polymerized or polymerized with UV-radiation. Exemplary monomers include, but are not limited to, aziridine, vinylamine, allylamine and oligomers from 80 g/mol to 1200 g/mol. Crosslinkers (e.g., 1,2-dichloroethane, epichlorohydrin, 1,6-diisocyanatohexane) could be used to crosslink oligomers.

### Substrates

The substrate can be formed from any desirable material, or combination of materials, suitable for use within the body. In some embodiments the substrate is formed from compliant and flexible materials, such as elastomers (polymers with elastic properties). Exemplary elastomers can be formed from various polymers including polyurethanes and polyurethane copolymers, polyethylene, styrene-butadiene copolymers, polyisoprene, isobutylene-isoprene copolymers (butyl rubber), including halogenated butyl rubber, butadiene-styrene-acrylonitrile copolymers, silicone polymers, fluorosilicone polymers, polycarbonates, polyamides, polyesters, polyvinyl chloride, polyether-polyester copolymers, polyether-polyamide copolymers, and the like. The substrate can be made of a single elastomeric material, or a combination of materials. Other materials for the substrate can include those formed of polymers, including oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, vinyl chloride, vinyl acetate, vinyl pyrrolidone, vinylidene difluoride, and styrene. Examples of condensation polymers include, but are not limited to, nylons such as polycaprolactam, polylauryl lactam, polyhexamethylene adipamide, and polyhexamethylene dodecanediamide, and also polyurethanes, polycarbonates, polyamides, polysulfones, poly(ethylene terephthalate), polydimethylsiloxanes, and polyetherketone.

Beyond polymers, and depending on the type of device, the substrate can also be formed of other inorganic materials such as metals (including metal foils and metal alloys), glass and ceramics.

Processes to modify substrates described above can include chemical modifications to improve performance characteristics of the substrate. Specific chemical processes that can be used include ozone treatment, chemical oxidation, acid chemical etching, base chemical etching, plasma treatment and corona treatment, surface grafting, thermally activated coating processes (both covalent and non-covalent) and surface modifications including coatings containing dopamine, tannic acid, plant polyphenols and other catechols or catechol containing derivatives of hydrophilic moieties. Additionally, processes to form substrates described above can include physical modifications for example, but not limited to, sand blasting and surface texturing (for example either during or after the molding process of polymers).

In some embodiments, the modification of substrates as described herein can allow for omission of a base coating layer (such as a hydrophilic layer) as substrate surfaces that have been modified will allow for improved adhesion of a hydrophobic therapeutic agent and cationic agent compared with that of a hydrophilic layer.

### Medical Devices

It will be appreciated that embodiments herein include, and can be used in conjunction with, various types of medical devices including, but not limited to, drug delivery devices such as drug eluting balloon catheters, drug-containing balloon catheters, stents, grafts, and the like.

Some embodiments described herein can be used in conjunction with balloon expandable flow diverters, and self-expanding flow diverters. Other embodiments can include uses in contact with angioplasty balloons (for example, but not limited to, percutaneous transluminal coronary angioplasty and percutaneous transluminal angioplasty). Yet other embodiments can include uses in conjunction with sinoplasty balloons for ENT treatments, urethral balloons and urethral stents for urological treatments and gastro-intestinal treatments (for example, devices used for colonoscopy).

Hydrophobic active agent can be transferred to tissue from a balloon-like inflatable device or from a patch-like device. Other embodiments of the present disclosure can further be used in conjunction with micro-infusion catheter devices. In some embodiments, micro-infusion catheter devices can be used to target active agents to the renal sympathetic nerves to treat, for example, hypertension.

Other exemplary medical applications wherein embodiments of the present disclosure can be used further encompass treatments for bladder neck stenosis (e.g. subsequent to transurethral resection of the prostrate), laryngotrachial stenosis (e.g. in conjunction with serial endoscopic dilatation to treat subglottic stenosis, treatment of oral cancers and cold sores and bile duct stenosis (e.g. subsequent to pancreatic, hepatocellular of bile duct cancer). By way of further example, embodiments herein can be used in conjunction with drug applicators. Drug applicators can include those for use with various procedures, including surgical procedures, wherein active agents need to be applied to specific tissue locations. Examples can include, but are not limited to, drug applicators that can be used in orthopedic surgery in order to apply active agents to specific surfaces of bone, cartilage, ligaments, or other tissue through physical contact of the drug applicator with those tissues. Drug applicators can include, without limitation, hand-held drug applicators, drug patches, drug stamps, drug application disks, and the like.

In use, various embodiments included herein can enable rapid transfer of therapeutic agents to specific targeted tissues. For example, in some embodiments, a care provider can create physical contact between a portion of a drug delivery device including a therapeutic agent and the tissue being targeted and the therapeutic agent will be rapidly transferred from the drug delivery device to that tissue. As such, precise control over which tissues the therapeutic agent is provided to can be achieved.

One beneficial aspect of various embodiments described herein is that the therapeutic agent can be transferred from the drug delivery device or coating to the targeted tissue very rapidly. In some embodiments substantial transfer of the therapeutic agent from the drug delivery device or coating to the tissue occurs in 30 minutes or less. In some embodiments substantial transfer of the therapeutic agent from the drug delivery device or coating to the tissue occurs in 15 minutes or less. In some embodiments substantial transfer of the therapeutic agent from the drug delivery device or coating to the tissue occurs in 10 minutes or less. In some embodiments substantial transfer of the therapeutic agent from the drug delivery device or coating to the tissue occurs in 5 minutes or less. In some embodiments substantial transfer of the therapeutic agent from the drug delivery device or coating to the tissue occurs in 2 minutes or less. In some embodiments substantial transfer of the therapeutic agent from the drug delivery device or coating to the tissue occurs in 1 minute or less.

Further aspects of exemplary components of coatings are described in U.S. Pat. Nos. 9,757,497; 10,213,529; and 11,147,902, the content of which is herein incorporated by reference.

Aspects may be better understood with reference to the following examples. These examples are intended to be representative of specific embodiments, but are not intended as limiting the overall scope of embodiments herein.

### EXAMPLES

### Example 1: Effect of Relative Humidity During Coating on Drug Transfer and Retention

A series of test substrates were coated with formulations consistent with embodiments herein (with microcrystalline sirolimus and PEI in the active agent layer) at different degrees of relative humidity during the coating process. The results show that the amount of the active agent transferred was maximized when coating operations were performed at between 20 to 40 percent relative humidity.

These examples are intended to be representative of specific embodiments but are not intended as limiting the overall scope of embodiments herein.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed, and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A drug delivery coating comprising:
a polymeric layer, the polymeric layer comprising a hydrophilic outer surface;
an active agent layer, wherein the active agent layer is disposed over the polymeric layer, the active agent layer comprising
a microcrystalline active agent; and
a cationic agent.

2. The drug delivery coating of claim 1, wherein the microcrystalline active agent has an average particle size of less than 50 μm.

3. The drug delivery coating of claim 1, wherein the microcrystalline active agent has an average particle size of less than 20 μm.

4. The drug delivery coating of claim 1, wherein the microcrystalline active agent is at least 95 percent crystalline.

5. The drug delivery coating of claim 1, wherein the total amount of amorphous active agent in the active agent layer is less than 5% by weight.

6. The drug delivery coating of claim 1, the microcrystalline active agent comprising sirolimus.

7. The drug delivery coating of claim 1, the cationic agent comprising at least one selected from the group consisting of polyethyleneimine (PEI), 1,2-dioleoyl-3-trimethylammonium-propane (DOTAP), and polyamidoamine dendrimers (PAMAM).

8. The drug delivery coating of claim 1,
wherein the microcrystalline active agent is arranged leaving gaps between some adjacent crystals; and
wherein the cationic agent fills at least some of the gaps.

9. The drug delivery coating of claim 8, wherein at least some gaps remain unfilled.

10. The drug delivery coating of claim 1, wherein the cationic agent coats at least some crystals of the microcrystalline active agent.

11. The drug delivery coating of claim 1, the polymeric layer further comprising a hydrophilic polymer.

12. The drug delivery coating of claim 11, the hydrophilic polymer comprising at least one selected from the group consisting of a methacrylamide and a polyvinylpyrrolidone.

13. The drug delivery coating of claim 11, the hydrophilic polymer comprising a methacrylamide copolymer.

14. The drug delivery coating of claim 11, the hydrophilic polymer comprising a photoreactive methacrylamide copolymer.

15. The drug delivery coating of claim 11, the hydrophilic polymer comprising poly[N-(3-aminopropyl)methacrylamide-co-N-(3-(4-benzoylbenazmido)propyl)methacrylamide].

16. The drug delivery coating of claim 1, the polymeric layer further comprising a photoreactive crosslinking agent.

17. The drug delivery coating of claim 16, the photoreactive crosslinking agent comprising ethylenebis (4-benzoylbenzyldimethylammonium) dibromide.

18. The drug delivery coating of claim 1, the polymeric layer further comprising a methacrylamide.

19. The drug delivery coating of claim 1, the polymeric layer further comprising a polyvinylpyrrolidone.

20. The drug delivery coating of claim 1, the active agent layer further an additive, the additive comprising at least one selected from the group consisting of glycogen, dextran, and F68 poloxamer.
